(19) United States
(12) Patent Application Publication
(10) Pub. No.: US 2025/0352282 A1
(43) Pub. Date: Nov. 20, 2025

(54) A SURGICAL ROBOTIC SYSTEM FOR EXECUTING A BONE IMPLANT SURGICAL PLAN

(71) Applicant: ECENTIAL ROBOTICS, GIERES (FR)

(72) Inventors: Lucie Thiebaut, Gières (FR); François Leitner, Gières (FR)

(21) Appl. No.: 18/871,270

(22) PCT Filed: May 9, 2024
(86) PCT No.: PCT/EP2024/062879

§ 371 Date: Dec. 3, 2024

(30) Foreign Application Priority Data
May 12, 2023 (EP) 23315199.2

(51) Int. Cl. A61B 34/30; A61B 17/32; A61B 34/10

(52) U.S. Cl. CPC A61B 34/30; A61B 17/320068; A61B 34/10; A61B 2034/102; A61B 2034/107; A61B 2034/305

(57) ABSTRACT

A surgical robotic system for executing a bone implant surgical plan—includes a surgical robot. The surgical robot includes a first robotic arm for manipulating a surgical tool during a surgical procedure at a surgical site. A control unit of the surgical robotic system includes a processor programmed to direct the surgical robot to position the surgical tool. The processor program includes instructions for executing an act of defining the location of the surgical tool with respect to the surgical site. The processor program also includes instructions for executing further acts of moving the surgical tool to a bone located within the surgical site, the location of which being predefined within the surgical plan, and of marking the bone with the surgical tool, the marking corresponding to the position of a bone implant to be implanted in the bone according to the surgical plan.

### CROSS-REFERENCE TO RELATED APPLICATIONS

This application is a national phase entry under 35 U.S.C. § 371 of International Patent Application PCT/EP2024/062879, filed May 9, 2024, designating the United States of America and published as International Patent Publication WO 2024/235827 A1 on Nov. 21, 2024, which claims the benefit, under Article 8 of the Patent Cooperation Treaty, of European Patent Application Serial No. 23315199.2, filed May 12, 2023.

### TECHNICAL FIELD

The present disclosure relates to a surgical robotic system for executing a bone implant surgical plan.

### BACKGROUND

A surgical robotic system is used for assisting a user (e.g., a surgeon) during a surgical intervention.

For example, in bone surgery, the user may have to implant one or several screws, or a bone implant, into a bone, such as a vertebra, a hip, a knee, etc.

In this regard, the use of a localization system that can localize trackers in real time may be used to carry out such manipulation, either in assistance of the surgeon or autonomously. Both the anatomical structure and the surgical tool can be localized, which allows determining in real time the relative positions of the surgical tool relative to the anatomical structure to be treated.

To that end, both the surgical tool and the anatomical structure may comprise a tracker rigidly attached thereto, each tracker being tracked by the localization system, therefore also called tracking system.

In some circumstances, a tracker may be rigidly attached to a part of the robotic system and not the surgical tool directly, allowing indirect localization of the tool based on knowledge at any time of the kinematic model of the robotic system between the tracker and the tool.

The surgical robotic system comprises a surgical robotic arm, having at least six degrees of freedom, and comprising an end effector generally holding a surgical tool. The surgical tool is to be placed with a given position and orientation relative to a surgical target. Once the surgical tool is placed relative to the surgical target, the surgical robotic system assists the surgeon in moving the surgical tool relatively to the surgical target. Thus, the surgeon may manoeuvre the surgical tool with the right position and orientation relative to the surgical target during the surgery, according to a surgical plan.

During a surgery such as a bone implant, the bone implant must be implanted with a very precise position relative to the bone.

To this end, it is known to implement ink pads, which are used manually to mark the bone where the bone implant is to be implanted. It is also known to use tracked stencil, which are manually adjusted on site to match the bone implant planned position, manually pinned down to the adjusted location, and manually used as a guide for a manual tool dedicated to imprint the bone implant negative into the bone.

These manual operations may lead to some imprecision of the relative position of the bone implant relative to the bone. Embodiments of the present disclosure aim to reduce or suppress this drawback.

### BRIEF SUMMARY

The present disclosure relates to a surgical robotic system for executing a bone implant surgical plan, the system comprising:
a surgical robot comprising a first robotic arm having at least six degrees of freedom for manipulating a surgical tool within a surgical site during a surgical procedure for implementing the surgical plan; and
a control unit, comprising a processor programmed to direct the surgical robot to position the surgical tool at the surgical site,
wherein the processor program includes instructions for executing the following step:
defining the location of the surgical tool with respect to the location of the surgical site, within a same three-dimensional coordinates system, in real-time.

It is essentially characterized in that:
the processor program includes instructions for further executing the following steps:
moving the surgical tool to a bone located within the surgical site, the location being predefined within the surgical plan; and
marking the bone with the surgical tool, the marking corresponding to the position of a bone implant to be implanted in the bone according to the surgical plan.

Advantageously, the surgical robotic system, according to the present disclosure, can fully execute a bone implant surgical planning, meaning without manual operation of a surgeon directly on a patient's body.

The position of a bone implant to be implanted in the bone can also be understood as the position of a planned bone implant anchoring system or keyed connection to be implanted in the bone.

It may be provided that the processor program includes instructions for moving the surgical tool within the surgical site according to the surgical plan or to the surgical implant planned position.

It may be provided that the surgical robot comprise the surgical tool.

It may be provided that the surgical tool is a passive surgical tool. For instance, it may be provided that the surgical tool is a stencil, which is inked with a biocompatible ink.

It may be provided that the surgical tool is motorized or not. For instance, it may be provided that the motorized tool is an electrical surgical tool or a pneumatical surgical tool, and, for instance, one amongst: an oscillating saw, a reciprocating saw, a Tuke saw, a drill.

For instance, it may be provided that the non-motorized tool is a chisel.

It may be provided that the surgical tool is an electrical scalpel.

Accordingly, the surgical tool may be understood as a cutting tool or a machining tool and indistinctively called “resecting tool.” Similarly, “cutting a bone” and “resecting a bone” shall be understood as indistinct expressions.

It may be provided that the processor program includes instructions for executing the following step: resecting the bone located within the surgical site with the surgical tool, the surgical tool being a resecting tool, such that marking the bone and resecting the bone can be implemented with the same surgical tool.

Resecting a bone creates a resection area on the bone.

It may be provided that the processor program includes instructions for resecting the bone located within the surgical site with the surgical tool such that the resected bone presents a resection area, such that the bone can support a bone implant.

The resection area, or cut area, may be a flat area.

It may be provided that the instructions for executing the step of marking the bone that shall support the bone implant with the surgical tool, further comprise instructions for executing, at the surgical site, at least on one of the following steps:
a. marking the bone that has been cut and that shall support the bone implant, on the cut area, and
b. marking the bone that has been cut and that shall support the bone implant away from the cut area.

It may be provided that the instructions for marking the bone with the surgical tool comprise marking on the bone the position, and, in particular, the central position, of a bone implant to be implanted in the bone, enabling keying in translation of the position of the implant.

It may be provided that the instructions for marking the bone with the surgical tool comprise marking on the bone the angular position of the bone implant, enabling keying in rotation of the position of the implant.

It may be provided that the bone implant comprises a set of at least two fins, the instructions for marking the bone with the surgical tool comprise marking on the bone the angular position of each fin of the set of fins.

It may be provided that the surgical robot further comprises a second robotic arm having at least six degrees of freedom for manipulating a surgical tool within a surgical site during a surgical procedure for implementing the surgical plan.

It may be provided that the control unit is located within the surgical robot.

It may be provided that the surgical site comprises a bone to receive the bone implant, the system further comprising a set of markers located on both the robotic arm and the bone to receive the bone implant; and a tracking system configured to locate the relative position of the robotic arm and the surgical site.

Preferably, markers are electromagnetic markers or optical markers, which can be identified by the localization system, which is further described.

Thanks to embodiments of the present disclosure, the surgeon can perfectly execute the surgeon's planning that, without the present disclosure, contains a purely manual step that does not guarantee the placement of the implant according to the surgical plan.

In the long term, embodiments of the present disclosure allow the surgeon to draw conclusions as to which planning leads to the best clinical outcome in which clinical indication. This type of analysis is impossible to date as the real positioning of the implant with respect to its planned positioning remains unknown.

With embodiments of the present disclosure, surgeons can collect data and find correlations leading to the best clinical outcome. Thus, surgeons can design efficient patient specific clinical course of actions to reach the best possible outcome for each patient.

When the present disclosure is embodied as a bone marking system leaving an engraved mark in the bone and not just a print, the mark (or marks) serves (respectively serve) as a further risk control measure to ensure the correct execution of the implant placement. Indeed, as such, the mark (or marks) in the bone drives (respectively drive) the positioning of the implant print not only visually but physically as well, enabling keying in translation and/or in rotation of the position of the implant.

Other features and advantages of the present disclosure will appear in the detailed description that is given as a mere illustrative and non-limitative example.

### BRIEF DESCRIPTION OF THE DRAWINGS

FIG. 1 illustrates a robotic arm according to the present disclosure, equipped with a set of trackers, in this case optical trackers, secured to a surgical tool;

FIG. 2 illustrates a robotic arm according to the present disclosure, mounted on a movable cart; and

FIG. 3 illustrates a tibia and a femur both resected and marked with a surgical robotic system according to the present disclosure.

### DETAILED DESCRIPTION

Surgical robotic systems are frequently used during surgical procedures to provide physicians with image-based information about a patient's anatomical situation and/or the position and orientation of a surgical instrument with respect to the patient.

Over the past decades, two-dimensional (2D) images and three-dimensional (3D) imaging techniques have become more and more implemented.

Typically, an imaging system, such as an X-ray imaging system, moves about a given trajectory (e.g., a simple rotation about an axis or a more complex trajectory) in order to obtain images from different projection angles. A 3D volume of a body region of interest is reconstructed from the images.

Other techniques such as “Bone Morphing” are known to construct a 3D mesh of a body region of interest.

Such body region of interest comprises or is a surgical site, which is defined as being a 3D volume where a surgical plan shall be implemented, and within which movements of a surgical tool are preferably constrained.

### Surgical Robotic System

A surgical robotic system according to embodiments of the present disclosure, may comprise:
a base, which may be a movable cart 200, or may be attached to an operating table,
a robotic arm 100 having a proximal end 130 extending from a base, and a distal end 140 opposite to the proximal end 130, the distal end 140 comprising a surgical tool 300, and
a control unit, comprising a processor programmed to direct the surgical robot to position the surgical tool at the surgical site, the controller being configured to controllably move the robotic arm 100 according to a planned trajectory of a surgical plan, to implement the surgical plan during a surgical procedure.

The surgical robotic system may comprise a surgical tool 300. In particular, the robotic arm 100 may be equipped with such surgical tool, which is further described.

The robotic arm 100 comprises a plurality of degrees of freedom in translation and/or rotation. Usually, the robotic arm 100 comprises at least five, and preferably six or seven, motorized degrees of freedom. To that end, the robotic arm 100 comprises a plurality of articulated segments 110 driven by motors. By convention, the segments 110 are numbered from the proximal end 130 (which is the end closest to the movable cart 200) to the distal end 140 (opposite the proximal end 130) of the robotic arm 100. The successive robotic arm 100 joints may be rotations or translations. Successive rotations may be orthogonal or parallel. Some parts of the robotic arm 100 may also use parallel mechanisms, such as a hexapod architecture.

The robotic system is active in the sense that it can hold and move a powered surgical tool that interacts directly with the anatomical structure, contrary to a passive robotic system that holds a guide in a predefined position with respect to the anatomical structure into which a powered surgical tool is inserted by a surgeon.

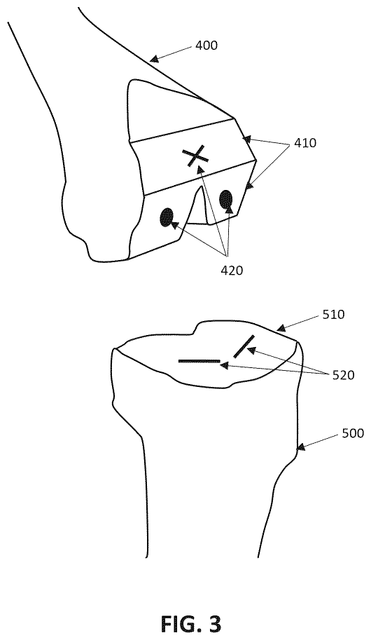

For example, a surgical saw is mounted on the robotic arm 100 end effector, as illustrated in FIG. 2, and actively marks, makes a notch or cuts the bone along a predefined path until an endpoint of a selected linear trajectory is reached, as illustrated in FIG. 3.

The robotic arm 100 is carried by the movable cart 200.

During a surgical operation, the cart 200 is intended to remain fixed relative to an operating table, on which a patient lies, while the robotic arm 100 is moved to reach surgical target(s) within the surgical site according to a surgical plan.

The robotic arm 100 comprises a plurality of degrees of freedom in translation and/or rotation.

The robotic arm 100 may be controlled in an autonomous mode according to desired targets and trajectories, manipulated using a collaborative mode (cobot), or telemanipulated using a master control device. A combination of two or more of these different modes may be used on the same robotic system.

The distal end 140 of the robotic arm 100 holds a surgical tool 300.

In one embodiment, the surgical tool 300 is a passive surgical tool. For instance, the surgical tool 300 is a stencil, which is inked with a biocompatible ink.

In another embodiment, the surgical tool 300 is a motorized surgical tool. For instance, the surgical tool 300 is one amongst: an oscillating saw, a reciprocating saw, a Tuke saw, an electrical scalpel, a drill.

The surgical robotic system also includes a control unit configured to controllably move the robotic arm 100.

The control unit comprises a processor, a data storage device and a communication device. The control unit may advantageously be embedded in the movable cart 200.

In other embodiments, the control unit may be provided separately from the movable cart 200 and may be configured to communicate wirelessly or by wires with the robotic arm 100.

### Localization System

To define the location of the surgical tool with respect to the location of the surgical site, within a same three-dimensional coordinates system, in real-time, a localization system may be provided, which is also indistinctively called “tracking system.”

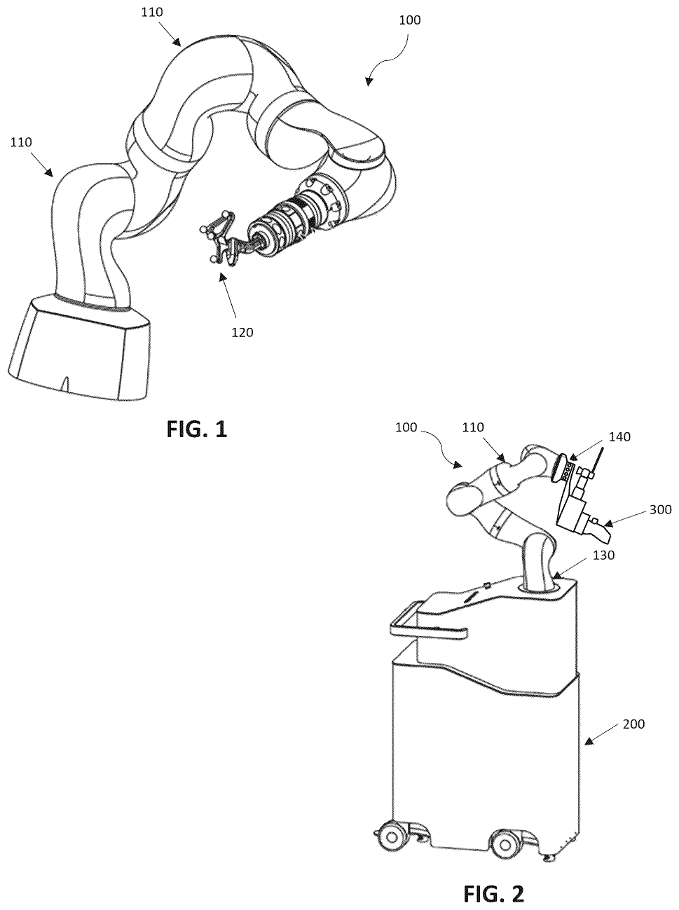

Typically, as known in itself, the localization system is configured to localize trackers 120, or indistinctively markers, which can be electromagnetic or optical markers, attached to the patient, to the robotic arm 100, and/or the surgical tool as illustrated in FIG. 1.

Optical markers can be passive optical markers, with a reflecting surface, or active optical markers, with a LED.

The localization system can localize position and orientation of any tracker 120 mounted rigidly on the bones at the surgical site and on the surgical tool 300 of the robotic system.

The localization system can be an optical system, or an electromagnetic system, or any combination of optical, electromagnetic, ultrasonic, inertia measurement devices and sensors, or passive electro-mechanical arms with encoders.

For instance, the localization system comprises a camera arranged to detect optical trackers 120, wherein each optical tracker 120 comprises a set of reflective markers, having, for example, a spherical shape.

Typically, a first set of at least one tracker 120 is rigidly attached to a bone within or adjacent to the surgical site. A second set of at least one tracker 120 is rigidly attached to the surgical tool 300 manipulated by the robotic arm 100.

The position and the orientation of the surgical tool 300 is known in a coordinate system attached to the second set of at least one tracker 120 at any time. Preferably, the position and the orientation of the surgical tool 300 is also known in a coordinate system attached to the first set of at least one tracker 120 at any time.

It is also possible to implement markerless solutions, such as shape recognition system using 3D scanners.

### Utilization of the Robotic System

The robotic system may be operated as follows.

At the beginning of surgery, a patient is equipped with a first tracker 120 (called “patient tracker”) detectable by the localization system.

A 3D image can be acquired either at the beginning of surgery using an X-ray imaging system itself (CBCT) or prior to surgery with another imaging system (CT or CBCT) and the 3D image is registered with respect to the patient tracker 120. Further techniques may be implemented for imagining the patient's surgical site, for instance bone morphing, 3D laser scanner or computer vision imagery with a stereo camera.

A plurality of trajectories of the surgical tool 300 are planned in the 3D image. For that purpose, a surgical planning software is used to define surgical targets interactively or automatically in the 3D image.

The robotic system, which may be mobile on the wheels of the cart 200 forming the base of the robotic arm 100, is brought near the surgical table, as illustrated in FIG. 2, where the surgical robotic system comprises a surgical tool 300.

The robotic system is equipped with a surgical tool 300, as previously described.

A second tracker 120 (called “robot tracker”) is mounted on the robotic arm 100, on the surgical tool 300 directly or on any sub-system of the robotic system. The calibration of the robot tracker 120 with respect to the surgical tool 300 can use several known calibration methods.

In a preferred embodiment, the position of the robot tracker 120 on the surgical tool 300 is reproducible and always the same, and a localized pointer is used to check that a particular point of the surgical tool 300 has precisely the expected coordinates with respect to the robot tracker 120.

In another preferred embodiment, the localized pointer is used to digitize at least three precisely defined points on the surgical tool 300 and a point-based calibration is applied.

The robotic system may be moved manually (cobot) or automatically, to align the surgical tool 300 to the planned trajectory, until it reaches an entry point on the bone surface at the surgical site.

Accordingly, the surgical tool 300 is moved to a bone located within the surgical site, the location being predefined within the surgical plan.

The robotic system can be servo-controlled on the movements of the patient tracker 120 to maintain alignment with the entry point position on the bone, compensating for any motions of the bone due to patient's breathing or any mechanical interactions.

Once the surgical tool 300 is located at the surgical site, the robotic arm 100 is controlled such that the surgical tool 300 is moved with respect to the bone in order to prepare the surgery according to the surgical plan.

In this case, the surgery is a bone implant. In this kind of surgery, the position of the bone implant relative to the position of the bone is decisive for the patient outcome.

According to the present disclosure, the surgical tool 300 is used to mark the bone with the surgical tool 300, the marking corresponding to the position of a bone implant to be implanted in the bone according to the surgical plan.

For instance, in one embodiment, a cross can be marked on the bone to indicate the localization of the central position of a bone implant to be implanted in the bone.

In another embodiment, which is combinable with the previous embodiment, the surgical tool 300 is used to mark on the bone the angular position of the bone implant to be implanted in the bone. For instance, the bone implant comprises a set of one or more fins and the surgical tool 300 is used to mark the angular position of each fin of the set of fins on the bone.

A surgical plan may require that the bone within the surgical site be resect.

In this case, advantageously, marking the bone and resecting the bone can be implemented with the same surgical tool 300, saving a lot of time as there is no need to replace a surgical tool 300 with another one.

When a bone within the surgical site is cut, the resecting area is a cutting area that may present a flat area. The bone may be partially or totally cut.

Thanks to embodiments of the present disclosure, the flat area of the bone once cut may be marked, as illustrated in FIG. 3.

In FIG. 3, a femur 400 has been cut. Accordingly, the femur 400 presents a resection area 410. In the illustrated case, the resection area 410 includes two resection areas 410, each of which being flat.

Thanks to embodiments of the present disclosure, is it possible to implement a surgical tool 300 (FIG. 2) to mark the bone. Accordingly, the resection area 410 of the femur 400 comprise a set of at least one mark 420, for implanting a femoral component bone implant. Advantageously, the shape of the mark 420 may be chosen by the surgeon, e.g. a cross, a line or a dot. Advantageously, the chosen shape of the mark 420 will ease the implant of the bone implant.

For instance, in FIG. 3, the tibia 500 comprises a resection area 510 that comprises two marks 520 presenting the shape of a line. Each line corresponds to the position of a respective fin of a bone implant to be implanted, enabling keying in rotation of the position of the implant.

But also, the bone (e.g., femur 400 and/or tibia 500) that has been cut may be marked away from the flat area (e.g., resection areas 410 and/or 510), depending on the surgical plan or on the bone implant to be implanted.

The resection area 410, 510 may be partly flat. Typically, the shape of the resection area 410, 510 is chosen based on the surgical operation to be implanted.

Advantageously, according to embodiments of the present disclosure, marking the bone is simpler than machining the bone, as machining the bone requires a complete knowledge of the 3D shape of the bone implant.

It may be provided that the surgical robotic system comprises two robotic arms 100. Preferably, the two robotic arms 100 are identical.

For instance, a first robotic arm 100 is equipped with a first surgical tool 300 and a second robotic arm 100 is equipped with a second surgical tool 300. The second surgical tool 300 may differ from the first surgical tool 300.

The first surgical tool 300 can be used for marking the bone as previously described, and the second surgical tool 300 can be used for the surgery.

Embodiments of the present disclosure may be implemented for bone implants, such as knee, hip, shoulder, ankle, and elbow implants.

### NOMENCLATURE

100 robotic arm
110 segment
120 tracker
130 proximal end
140 distal end
200 cart
300 surgical tool
400 femur
410 resection area of the femur
420 mark on the femur, for implanting a femoral component bone implant
500 tibia
510 resection area of the tibia
520 mark on the tibia, for implanting a tibial component bone implant

1. A surgical robotic system for executing a bone implant surgical plan, the system comprising:
a surgical robot comprising a robotic arm having at least six degrees of freedom for manipulating a surgical tool within a surgical site during a surgical procedure for implementing the surgical plan; and
a control unit comprising a processor programmed to direct the surgical robot to position the surgical tool at the surgical site,
wherein the processor program includes instructions for executing acts comprising:
defining a location of the surgical tool with respect to a location of the surgical site, within a same three-dimensional coordinates system, in real-time;
moving the surgical tool to a bone located within the surgical site, a location of the bone being predefined within the surgical plan; and
marking the bone with the surgical tool, the marking corresponding to a position of a bone implant to be implanted in the bone according to the surgical plan.

2. The surgical robotic system of claim 1, wherein the surgical robot further comprises the surgical tool, the surgical tool being a passive surgical tool.

3. The surgical robotic system of claim 2, wherein the passive surgical tool is a stencil, the stencil being inked with a biocompatible ink.

4. The surgical robotic system of claim 1, wherein the surgical robot further comprises the surgical tool, the surgical tool being a motorized surgical tool.

5. The surgical robotic system of claim 4, wherein the motorized surgical tool is an electrical surgical tool.

6. The surgical robotic system of claim 5, wherein the processor program includes additional instructions for executing an additional act of cutting the bone located within the surgical site with the electrical surgical tool, the electrical surgical tool being a cutting tool, the marking of the bone and the cutting of the bone being implementable with a same surgical tool.

7. The surgical robotic system of claim 6, wherein the additional instructions of the processor program for executing the cutting of the bone located within the surgical site with the electrical surgical tool are defined such that executing the cutting configures the bone to support the bone implant.

8. The surgical robotic system of claim 6, wherein the instructions for executing the marking of the bone with the surgical tool comprise instructions for executing, at the surgical site after executing the cutting of the bone to provide a cut area, at least-on one marking act chosen from:
marking, on the cut area, the bone that has been cut and that shall support the bone implant; and
marking, away from the cut area, the bone that has been cut and that shall support the bone implant.

9. The surgical robotic system of claim 1, wherein the instructions for executing the marking of the bone with the surgical tool comprise instructions for executing marking, on the bone, a central position of the bone implant to be implanted in the bone to enable keying in translation of the position of the bone implant.

10. The surgical robotic system of claim 1, wherein the instructions for executing the marking of the bone with the surgical tool comprise instructions for executing marking, on the bone, an angular position of the bone implant to be implanted in the bone to enable; keying in rotation of the position of the bone implant.

11. The surgical robotic system of claim 10, wherein the bone implant comprises a set of at least two fins, the instructions for executing the marking, on the bone, of the angular position of the bone implant comprising instructions for executing marking, on the bone, of the angular position of each fin of the set of at least two fins.

12. The surgical robotic system of claim 1, wherein the surgical robot further comprises an additional robotic arm having at least six degrees of freedom for manipulating an additional surgical tool within the surgical site during the surgical procedure for implementing the surgical plan.

13. The surgical robotic system of claim 1, wherein the control unit is located within the surgical robot.

14. The surgical robotic system of claim 1, wherein the surgical site comprises the bone to receive the bone implant, the system further comprising a set of markers located on both the robotic arm and the bone to receive the bone implant; and a tracking system configured to locate a relative position of the robotic arm and the bone at the surgical site.

15. The surgical robotic system of claim 5, wherein the electrical surgical tool is a cutting tool or a machining tool.

16. The surgical robotic system of claim 5, wherein the electrical surgical tool comprises one electrical surgical tool chosen from an oscillating saw, a reciprocating saw, a Tuke saw, a drill, and an electrical scalpel.

17. The surgical robotic system of claim 7, wherein the instructions for executing the marking of the bone with the surgical tool comprise instructions for executing, at the surgical site after executing the cutting of the bone to provide a cut area, at least one marking act chosen from:
marking, on the cut area, the bone that has been cut and that shall support the bone implant; and
marking, away from the cut area, the bone that has been cut and that shall support the bone implant.
